(19) United States
(12) Patent Application Publication
(10) Pub. No.: US 2013/0060147 A1
(43) Pub. Date: Mar. 7, 2013

(54) OCCLUSIVE NON-INFLATABLE BLOOD PRESSURE DEVICE

(71) Applicants: James P. Welch, Mission Viejo, CA (US); Brian Spencer Long, Aliso Viejo, CA (US)

(72) Inventors: James P. Welch, Mission Viejo, CA (US); Brian Spencer Long, Aliso Viejo, CA (US)

(73) Assignee: MASIMO CORPORATION, Irvine, CA (US)

(21) Appl. No.: 13/565,691

(22) Filed: Aug. 2, 2012

Related U.S. Application Data
(60) Provisional application No. 61/515,033, filed on Aug. 4, 2011.

(51) Int. Cl. A61B 5/0205; A61B 5/11; A61B 6/00; A61B 5/022; A61B 5/025

(52) U.S. Cl. 600/479; 600/493; 600/483; 600/499

(57) ABSTRACT

A blood pressure device including a compressible material can be placed around a limb of a patient (e.g. a band or cuff). Further, the blood pressure device can include a sleeve that, at least partially, covers the compressible material and is capable of compressing the compressible material to occlude a patient's blood vessel without inflating the sleeve. In some embodiments, the sleeve can compress the compressible material using a motor assembly. This motor assembly can include a motor and any additional mechanical devices that can be used to facilitate compressing the compressible material. For example, the motor assembly may include one or more of the following: a cable, a pulley, and a gear assembly, such as a worm drive or any other gear assembly that can facilitate compressing the compressible material.

### RELATED APPLICATION

This application claims the benefit of priority under 35 U.S.C. §119(e) of U.S. Provisional Patent Application No. 61/515,033, filed on Aug. 4, 2011, and entitled “OCCLUSIVE NON-INFLATABLE BLOOD PRESSURE DEVICE,” the disclosure of which is hereby incorporated by reference in its entirety.

### BACKGROUND

Hospitals, nursing homes, and other patient care facilities typically include patient monitoring devices at one or more bedsides in the facility. Patient monitoring devices generally include sensors, processing equipment, and displays for obtaining and analyzing a medical patient's physiological parameters, such as blood oxygen saturation level, respiratory rate, and the like. Clinicians, including doctors, nurses, and other medical personnel, use the physiological parameters obtained from patient monitors to diagnose illnesses and to prescribe treatments. Clinicians also use the physiological parameters to monitor patients during various clinical situations to determine whether to increase the level of medical care given to patients.

Blood pressure is one example of a physiological parameter that can be monitored. Many devices allow blood pressure to be measured by sphygmomanometer systems that utilize an inflatable cuff applied to a person's arm. The cuff is inflated to a pressure level high enough to occlude a major artery. When air is slowly released from the cuff, blood pressure can be estimated by detecting “Korotkoff” sounds using a stethoscope placed over the artery.

### SUMMARY

In certain embodiments, a blood pressure device includes a blood pressure cuff, an acoustic sensor, and a pressure sensor. The blood pressure cuff can selectively occlude and de-occlude a blood vessel of a patient without inflation of the blood pressure cuff. Further, the acoustic sensor can detect a biological sound of the patient responsive to de-occlusion of the blood vessel, the biological sound reflecting a measurement time at which a blood pressure measurement should be taken. In a number of implementations, the pressure sensor can output a pressure signal responsive to actuation of the blood pressure cuff. In a number of implementations, the pressure signal at the measurement time is indicative of a blood pressure of the patient.

In certain embodiments, a blood pressure device includes a compressible material, a sleeve, and a pressure sensor. The compressible material may be placed around a limb of a patient. Further, the sleeve may be disposed at least partially around the compressible material and may compress the compressible material. The pressure sensor can output a pressure signal responsive to compression of the compressible material. In certain implementations, the pressure signal reflects a blood pressure of the patient.

In certain embodiments, a system using, for example, a blood pressure device can cause a non-inflatable blood pressure cuff to occlude a blood vessel of a patient. Further, the system can cause the non-inflatable blood pressure cuff to de-occlude the blood vessel subsequent to said occlusion. Moreover, the system can detect, with an acoustic sensor, a biological sound responsive to said de-occlusion of the blood vessel. In certain embodiments, the system can take a blood pressure reading responsive to detection of the biological sound.

In some embodiments, a blood pressure device includes a compressible material and a pressure sensor. The compressible material can be placed around a limb of a patient and selectively compressed without inflation to cause selective occlusion of a blood vessel. Further, the pressure sensor can output a pressure signal responsive to compression of the compressible material. In some implementations, the pressure signal reflects a blood pressure of the patient.

### BRIEF DESCRIPTION OF THE DRAWINGS

Various embodiments will be described hereinafter with reference to the accompanying drawings. These embodiments are illustrated and described by example only, and are not intended to limit the scope of the disclosure. In the drawings, similar elements have similar reference numerals.

FIG. 1 illustrates an embodiment of a patient monitoring system.

FIG. 2 illustrates another embodiment of a patient monitoring system.

FIG. 3 illustrates an embodiment of a cuff measurement and control system.

FIGS. 4A-4B illustrate embodiments of a blood pressure cuff.

FIGS. 5A-5B illustrate top and bottom perspective views, respectively, of an acoustic sensor.

FIG. 6 illustrates another embodiment of a patient monitoring system.

FIG. 7 illustrates a flow diagram for one embodiment of a blood pressure measurement process.

FIG. 8 illustrates an embodiment of a calibration curve.

FIG. 9 illustrates an embodiment of a process for triggering an occlusive blood pressure measurement.

FIG. 10 illustrates another embodiment of a cuff measurement and control system.

FIG. 11 illustrates a flow diagram for one embodiment of a sleep-related illness detection process.

### DETAILED DESCRIPTION

In out-patient settings, blood pressure is often measured by a healthcare worker (e.g. a nurse), who uses an inflatable blood pressure cuff to facilitate determining a patient's blood pressure. When the healthcare worker is well-trained, it is unlikely that the inflatable blood pressure cuff will burst or harm the patient. However, a number of situations exist where the inflatable blood pressure cuff could potentially burst harming the patient. For example, the healthcare worker may not be well-trained or may be over-worked and may inadvertently over-inflate the blood pressure cuff. Further, a number of situations exist where the blood pressure measurement is obtained by an automated system, which may not always be monitored when blood pressure is being measured. For example, a patient in a hospital may have an automated blood pressure measurement system or an oscillatory blood pressure measurement system attached for an extended period of time. The unmonitored or semi-monitored blood pressure cuff could burst due to over-inflation, age, or excessive use potentially harming the patient.

This disclosure describes embodiments of a blood pressure device that can obtain a blood pressure reading from a patient without inflation. In one embodiment, the blood pressure device includes a compressible material that can be placed around a limb of a patient (e.g. a band or cuff). Further, the blood pressure device can include a sleeve that, at least partially, covers the compressible material and is capable of compressing the compressible material to occlude a patient's blood vessel without inflating the sleeve. In some embodiments, the sleeve can compress the compressible material using a motor assembly. This motor assembly can include a motor and any additional mechanical devices that can be used to facilitate compressing the compressible material. For example, the motor assembly may include one or more of the following: a cable, a pulley, and a gear assembly, such as a worm drive or any other gear assembly that can facilitate compressing the compressible material. In one embodiment, the blood pressure device occludes the patient's blood vessel without using a pneumatic device or mechanism.

### First Example of a Patient Monitoring System

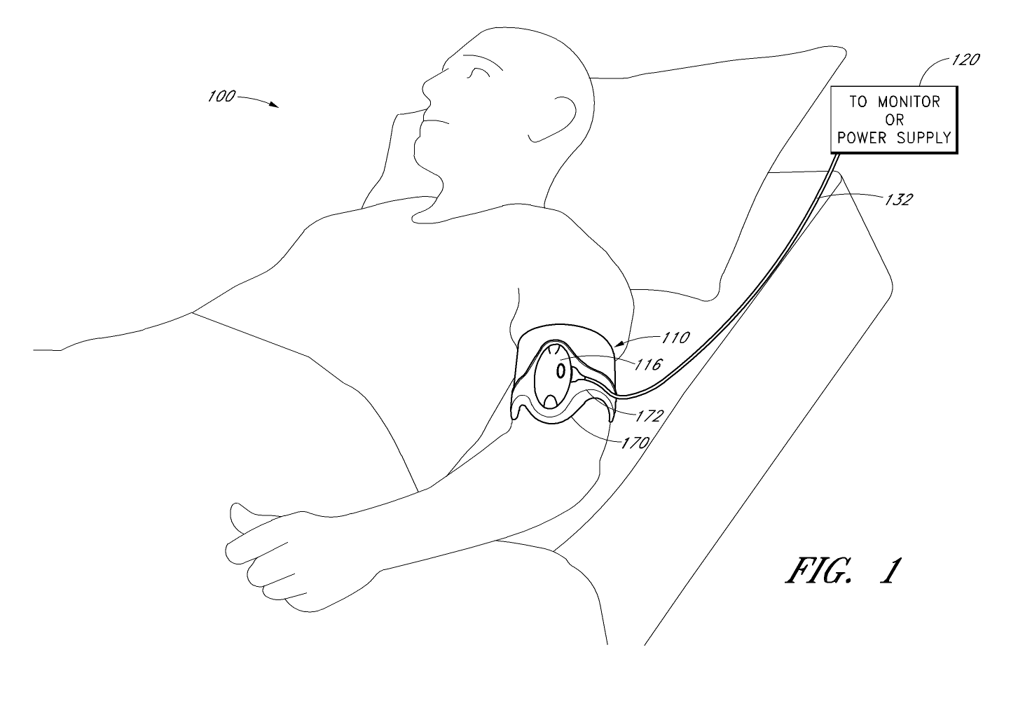

FIG. 1 illustrates an embodiment of a patient monitoring system 100. The patient monitoring system 100 includes a non-inflatable cuff 110 with a patient device 116 for providing physiological information to a monitor 120 or which can receive power from a power supply (120). The non-inflatable cuff 110 can be a blood pressure cuff that includes a compressible material 170 and a sleeve 172. The patient device 116 may be coupled via cable 132 to the monitor 120. Alternatively, the patient device 116 may communicate wirelessly with the monitor 120. In some embodiments, the patient device 116 includes processing capability, allowing the patient device 116 to implement at least some of the functions of the patient monitor 120. The separate monitor 120 (or external power supply) can therefore be omitted in some embodiments.

The compressible material 170 can include any material that can be compressed against a patient to occlude the patient's blood vessel. For example, the compressible material 170 can be a gel bladder, a cloth, a foam pad, or any other non-rigid material that can be safely compressed against a patient to occlude the patient's blood vessel. Further, in some embodiments, the compressible material 170 may include both compressible portions and non-compressible portions. For example, the portion that is placed over the blood vessel to be occluded may be compressible, while the remainder may not. Additionally, the compressible material 170 may be disposable, reusable, or resposable. Resposable devices generally include devices, components, or materials that are partially reusable and partially disposable.

The sleeve 172 can include any material or device that can at least partially overlap the compressible material 170 and that is capable of compressing, at least partially, the compressible material 170. The placement of the sleeve 172 with respect to the compressible material 170 is not limited so long as the sleeve 172 can compress the compressible material 170. For example, the sleeve 172 may, at least in part, be placed around the compressible material 170. As a second example, the sleeve 172 may, at least in part, be slipped over the compressible material 170.

Further, the sleeve 172 can include a motor assembly (not shown) that facilitates the compression of the compressible material 170. The motor assembly can include a motor and any number of additional devices for compressing the compressible material 170. For example, the motor assembly can include one or more of the following: a cable, a pulley, and a gear assembly, such as a worm drive or any other gear assembly that can facilitate compressing the compressible material. In some embodiments, the motor assembly can tighten the sleeve 172 against the compressible material 170 causing the compressible material 170 to compress against a patient, which in turn causes the cuff 110 to occlude a blood vessel of the patient.

Although the cuff 110 is depicted in FIG. 1 around a patient's upper left arm, it is possible for the compressible material 170 to be placed around the patient's right arm, the patient's leg, a different portion of the patient's arm, or any other portion of the patient's body that can provide a blood pressure reading. Further, it is possible for the cuff 110 to be placed in contact with any portion of the patient's body that can provide a blood pressure reading. In addition, although the cuff 110 is illustrated in the shape of an arm-band, it is possible for the cuff 110 to be shaped differently for use with different part's of the body. For example, the cuff 110 may be shaped like a rectangular strip or patch for use on a patient's head or torso.

The patient device 116 can include any device that can present physiological information to a healthcare worker or can transmit the physiological information to the monitor 120, which can then present the physiological information to the healthcare worker. In some embodiments, the patient device 116 can control the operation of the cuff 110. Alternatively, the monitor 120 may control the operation of the cuff 110. In some cases, the patient monitoring system 100 may include both the patient device 116 and the monitor 120. Alternatively, the patient monitoring system 100 may include one of the patient device 116 and the monitor 120, but not the other. In some instances, the patient device 116 may also be a monitor.

### Second Example of a Patient Monitoring System

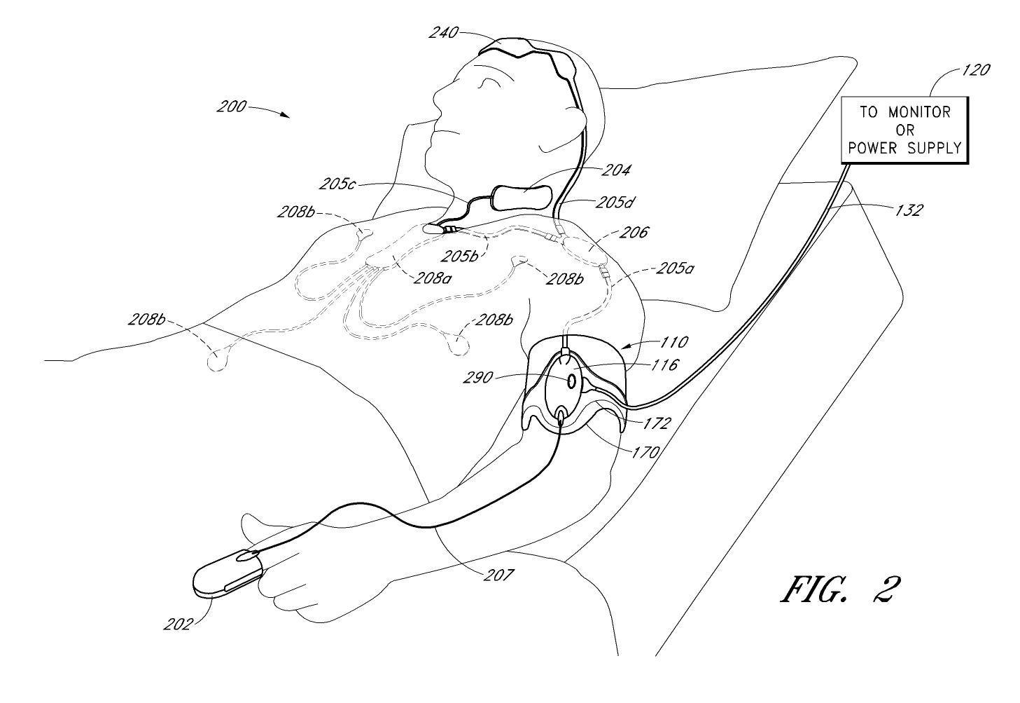

FIG. 2 illustrates another embodiment of a patient monitoring system 200. The features of the patient monitoring system 200 can be combined with any of the features of the systems described above. Likewise, any of the features described above can be incorporated into the patient monitoring system 200. Further, elements of FIG. 2 that share reference numerals with elements of FIG. 1 may be configured similarly. FIG. 2 shows the cuff 110 of FIG. 1 in the context of a multi-parameter patient monitoring system 200.

Advantageously, in the depicted embodiment, the patient monitoring system 200 includes a cable hub 206 that enables one or many sensors to be selectively connected and disconnected to the cable hub 206. Further, the patient device 116 is coupled with the cable hub 206 via a cable 205a. The cable hub 206 can be selectively connected to one or more sensors. In the depicted embodiment, example sensors shown coupled to the cable hub 206 include an electrocardiograph (ECG) sensor 208a and a brain sensor 240. The ECG sensor 208a can be a single-lead or a multi-lead sensor. The brain sensor 240 can be an electroencephalography (EEG) sensor and/or an optical sensor. An example of an EEG sensor that can be used as the brain sensor 240 is the SEDLine™ sensor available from Masimo® Corporation of Irvine, Calif., which can be used for depth-of-anesthesia monitoring among other uses. Optical brain sensors can perform spectrophotometric measurements using, for example, reflectance pulse oximetry. The brain sensor 240 can incorporate both an EEG/depth-of-anesthesia sensor and an optical sensor for cerebral oximetry.

The ECG sensor 208a is coupled to an acoustic sensor 204 and one or more additional ECG leads 208b. For illustrative purposes, four additional leads 208b are shown, for a 5-lead ECG configuration. In other embodiments, one or two additional leads 208b are used instead of four additional leads. In still other embodiments, up to at least 12 leads 208b can be included. Acoustic sensors can also be disposed in the ECG sensor 208a and/or lead(s) 208b or on other locations of the body, such as over a patient's stomach (e.g., to detect bowel sounds, thereby verifying patient's digestive health, for example, in preparation for discharge from a hospital). Further, in other embodiments, the acoustic sensor 204 can connect directly to the cable hub 206 instead of to the ECG sensor 208a.

Additionally, in some embodiments, the patient device 116 is coupled with an optical sensor 202 via cable 207. Although depicted as a fingertip sensor, the optical sensor 202 can be used with any part of a patient to obtain physiological information. For example, the optical sensor 202 can be placed on a toe or an ear. The optical sensor 202 can include one or more emitters and detectors for obtaining physiological information indicative of one or more blood parameters of the patient. These parameters can include various blood analytes such as oxygen, carbon monoxide, methemoglobin, total hemoglobin, glucose, proteins, lipids, a percentage thereof (e.g., concentration or saturation), and the like. The optical sensor 202 can also be used to obtain a photoplethysmograph (PPG), a measure of plethysmograph variability, a measure of blood perfusion, and the like.

In some embodiments, the optical sensor 202 can be used to detect reduced pulsatile blood flow in an extremity of the patient. This reduction in pulsatile blood flow can be used to determine whether a blood vessel is occluded. Further, in some cases, the reduction in pulsatile blood flow can be used to determine a degree to which the blood vessel is occluded. The cuff 110 can de-occlude the blood vessel in response to the detected reduction in pulsatile blood flow. For example, in response to determining that the pulsatile blood flow is substantially zero, indicating that the blood vessel is substantially or completely occluded, the cuff 110 can cause the sleeve 172 to reduce, at least in part, the amount of compression applied to the compressible material 170.

As mentioned above, the cable hub 206 can enable one or many sensors to be selectively connected and disconnected to the cable hub 206. This configurability aspect of the cable hub 206 can allow different sensors to be attached or removed from a patient based on the patient's monitoring needs, without coupling new cables to the monitor 120. Instead, a single, light-weight cable 132 couples to the monitor 120 in certain embodiments, or wireless technology can be used to communicate with the monitor 120. A patient's monitoring needs can change as the patient is moved from one area of a care facility to another, such as from an operating room or intensive care unit to a general floor. The cable configuration shown, including the cable hub 206, can allow the patient to be disconnected from a single cable to the monitor 120 and easily moved to another room, where a new monitor can be coupled to the patient. Of course, the monitor 120 may move with the patient from room to room, but the single cable connection 132 rather than several can facilitate easier patient transport.

Further, in other embodiments, the patient device 116 may be optional. In such embodiments, the cable hub 206 and/or the cuff 110 can instead connect directly to the monitor 120, either wirelessly or via a cable. Additionally, the cable hub 206 or the patient device 116 may include electronics for front-end processing, digitizing, or signal processing for one or more sensors. Placing front-end signal conditioning and/or analog-to-digital conversion circuitry in one or more of these devices can make it possible to send continuous waveforms wirelessly and/or allow for a small, more user-friendly wire (and hence cable 132) routing to the monitor 120.

The cable hub 206 can also be attached to the patient via an adhesive, allowing the cable hub 206 to become a wearable component. Together, the various sensors, cables, and cable hub 206 shown can be a complete body-worn patient monitoring system. The body-worn patient monitoring system can communicate with a patient monitor 120 as shown, which can be a tablet, handheld device, a hardware module, or a traditional monitor with a large display, to name a few possible devices.

Further, in some embodiments, the patient device 116 can include a wellness monitor 290 that can provide patient status information to a healthcare worker. In some embodiments, the wellness monitor 290 enables users, who may or may not be trained in using a patient monitoring system, to check the status of a patient without reviewing each possible physiological parameter that the patient monitoring system may be capable of presenting. Advantageously, in some embodiments, the use of the wellness monitor 290 enables a healthcare worker, or other user, to monitor a greater number of patients in a shorter period of time than with systems that do not include a wellness monitor 290. For example, a healthcare worker, in some embodiments, may be able to determine the status of a patient from the door of the patient's room, or from a central floor monitor, without entering the patient's room or checking each physiological parameter.

In some embodiments, the wellness monitor 290 can use any technique that can present a patient's status to a user in a clear unambiguous manner in a shorter amount of time than traditional systems that do not include a wellness monitor 290. For example, the wellness monitor 290 can include a status light that can indicate when the patient's blood pressure satisfies a threshold (e.g. a green light when the blood pressure satisfies a first threshold, a yellow light when the blood pressure satisfies a second threshold, a red blinking light when the blood pressure satisfies a third threshold, and a solid red light when the blood pressure satisfies a fourth threshold). As a second example, the wellness monitor 290 can provide an auditory status when the patient's blood pressure satisfies a threshold (e.g. the third and/or fourth threshold of the previous example).

Further, the wellness monitor 290 may indicate when a set of parameters satisfy a threshold. For example, a green light and lack of auditory signal may indicate that all monitored parameters are within healthy levels. A yellow light may indicate that one or more monitored parameters should be monitored more closely. Alternatively, a yellow light may indicate that a sensor is no longer receiving a physiological signal. For example, the sensor may have become disconnected or may be malfunctioning. A red light and/or a loud auditory signal may indicate that one or more monitored parameters deviate significantly from acceptable levels.

Additional examples of various types of wellness monitors that can be used herein are described in the following provisional applications, each of which is hereby incorporated by reference in its entirety: U.S. Provisional Application No. 61/442,264, filed Feb. 13, 2011, titled “Complex System Characterizer,” U.S. Provisional Application No. 61/393,869, filed Oct. 15, 2010, titled “DNA Risk Analysis System,” and U.S. Provisional Application No. 61/391,067, filed Oct. 7, 2010, titled “Risk Analysis System.”

Additional examples of patient monitoring systems that can be used herein are described in U.S. application Ser. No. 13/010,653, filed Jan. 20, 2011, titled “Wireless Patient Monitoring System,” and in U.S. application Ser. No. 12/840,209, filed Jul. 20, 2010, titled “Wireless Patient Monitoring System,” both of which are hereby incorporated by reference in their entirety.

### Example of a Cuff Measurement and Control System

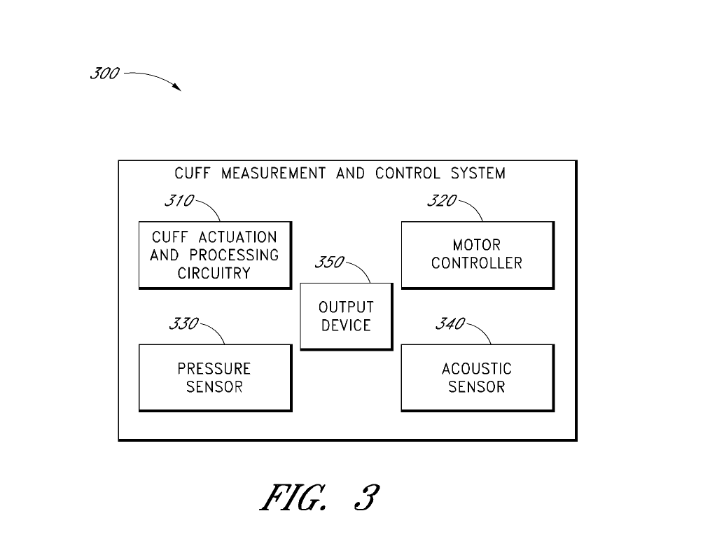

FIG. 3 illustrates an embodiment of a cuff measurement and control system 300. The cuff measurement and control system 300 can include any system for controlling a blood pressure cuff (e.g. cuff 110) and for obtaining, using the blood pressure cuff, physiological information to be provided to a healthcare worker and/or to another system, such as the monitor 120. Generally, the cuff measurement and control system 300 can be included as part of a blood pressure cuff, such as cuff 110. Further, the cuff measurement and control system 300 can be a separate component or may be included as part of a patient device (e.g. the patient device 116). In addition, one or more of the components of the cuff measurement and control system 300 may be included as part of a patient monitor (e.g. monitor 120).

The cuff measurement and control system 300 can include a number of subsystems including: cuff actuation and processing circuitry 310, a motor controller 320, a pressure sensor 330, an acoustic sensor 340, and an output device 350.

The cuff actuation and processing circuitry 310 can generally include any circuitry or processor for determining when to obtain a blood pressure reading and for actuating the blood pressure cuff. This determination can be based on readings from one or more sensors, such as from the optical sensor 202. Alternatively, the cuff actuation and processing circuitry 310 may determine when to obtain the blood pressure reading based on time. For example, the cuff actuation and processing circuitry 310 can be configured to actuate the blood pressure cuff and obtain a blood pressure reading every hour. In some cases, the cuff actuation and processing circuitry 310 may actuate the cuff and obtain a blood pressure reading in response to a manual input; for example, via a button on the blood pressure cuff 110 or via a signal from the monitor 120 provided in response to an action by a healthcare worker.

In some embodiments, the cuff actuation and processing circuitry 310 can include signal conditioning circuitry. For example, the cuff actuation and processing circuitry 310 may include circuitry for converting analog signals into digital signals or for signal filtering, such as for removing noise from the signal. Alternatively, in some embodiments, the signal conditioning circuitry may be included with the monitor 120 or may be part of a distinct or separate system.

In some embodiments, the cuff actuation and processing circuitry 310 includes both signal conditioning capabilities and some or all of the capabilities of the monitor 120. For example, in some cases the cuff actuation and processing circuitry 310 is capable of actuating the motor assembly, via motor controller 320, converting and filtering signals obtained via one or more of the pressure sensor 330 and the acoustic sensor 340, and causing one or more physiological readings to be presented to a user via the output device 350. In alternative embodiments, the cuff actuation and processing circuitry 310 may be limited to signal processing. In such cases, the monitor 120 may be used to actuate the cuff 110 and to present physiological information or readings to the user. In some embodiments, the cuff actuation and processing circuitry 310 may also be capable of monitoring one or more physiological readings. Based on these physiological readings, the cuff actuation and processing circuitry 310 can actuate the cuff 110 to obtain a blood pressure reading and/or alert a user, such as via the wellness monitor 290 or an alarm.

Further, the cuff actuation and processing circuitry 310 can include circuitry for determining a value associated with a pre-defined value-set based on signals received from one or more sensors (e.g. the pressure sensor 330 or the acoustic sensor 340). For example, the cuff actuation and processing circuitry can determine a blood pressure value based, at least in part, on a pressure reading from the pressure sensor 330.

The cuff actuation and processing circuitry 310 can actuate and de-actuate the blood pressure cuff using the motor controller 320. The motor controller 320 can include any system for activating and controlling a motor associated with the cuff (e.g. the cuff 110) to tighten a sleeve 172 around the compressible material 170 to occlude a patient's blood vessel. Activating and controlling the motor can include controlling one or more operating characteristics associated with the motor. For example, the motor controller 320 can activate or deactivate the motor, select the direction of rotation of the motor, regulate the speed of the motor, or regulate the torque of the motor, among other options.

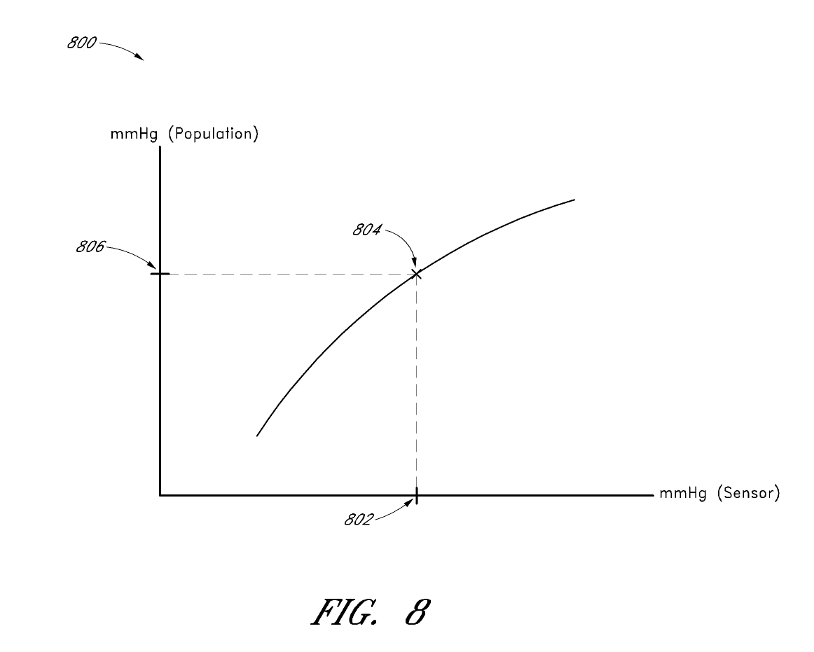

To obtain the blood pressure reading, the cuff actuation and processing circuitry 310 can use readings obtained from one or more sensors. For example, the cuff actuation and processing circuitry 310 can use one or more of the pressure sensor 330 and the acoustic sensor 340 to obtain the blood pressure reading. The pressure sensor 330 can include any type of sensor that can be used to measure the pressure applied by the sleeve 172 to the compressible material 170. For example, the pressure sensor 330 can include a strain gauge or a piezoelectric sensor. The acoustic sensor 340 can include any type of sensor that can be used to measure biological sounds. The process of obtaining the blood pressure reading is described in more detail below with respect to FIG. 7.

Optionally, the cuff measurement and control system 300 includes the output device 350. The output device 350 can present physiological information obtained by the blood pressure cuff. Further, the output device 350 can include a wellness monitor as described above with respect to FIG. 2. Alternatively, the monitor 120 may present the physiological information. Further, instead of, or in addition to, the output device 350 including the wellness monitor, the monitor 120 may include the wellness monitor.

### Examples of Blood Pressure Cuffs

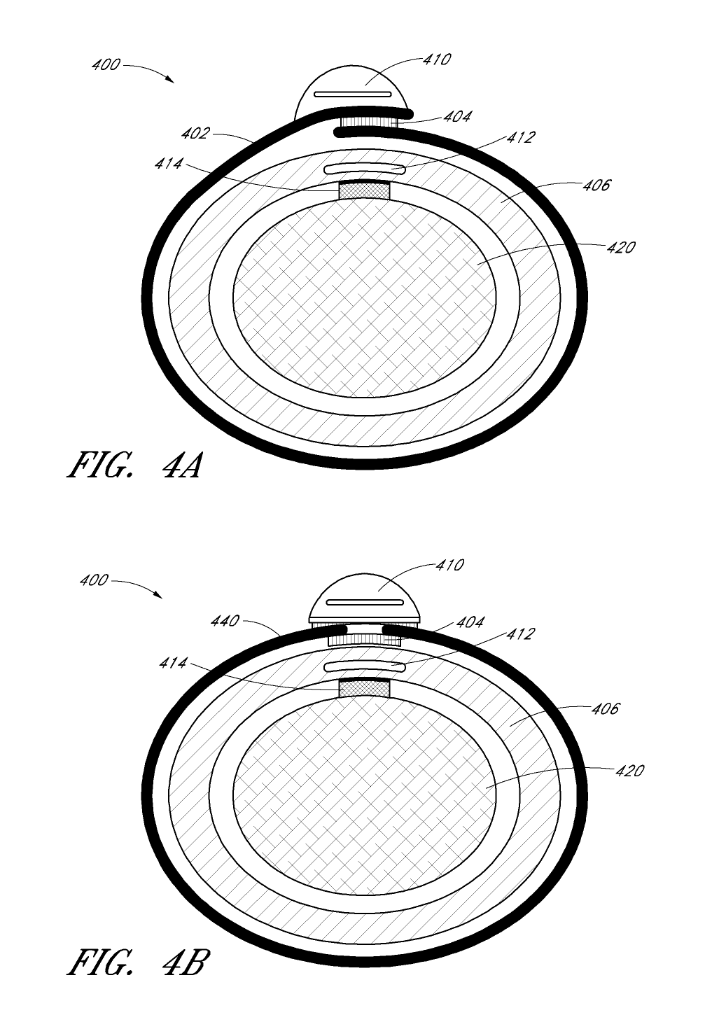

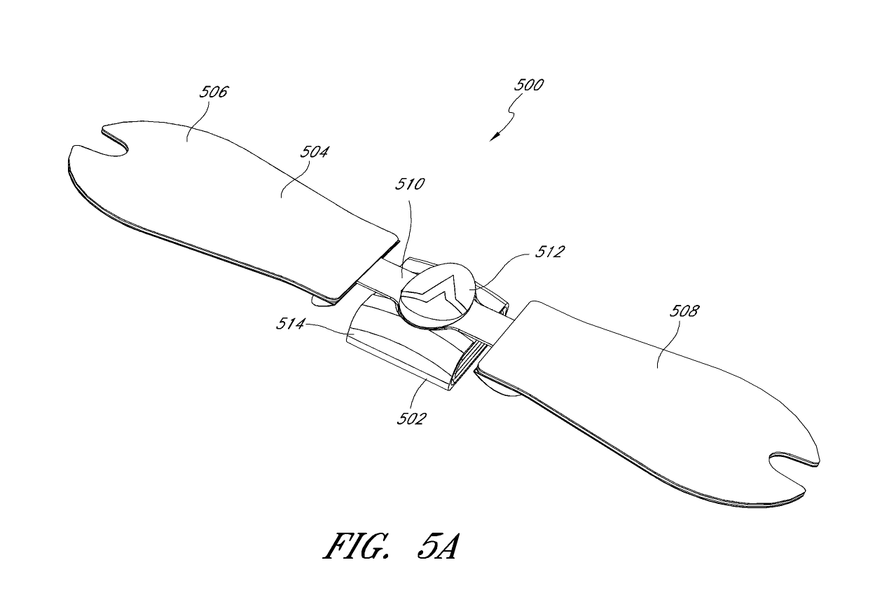

FIGS. 4A-4B illustrate embodiments of non-inflatable blood pressure cuffs. FIG. 4A illustrates an embodiment of a non-inflatable blood pressure cuff 400 with an overlapping sleeve 402 that at least partially covers or overlaps a compressible material 406. The non-inflatable blood pressure cuff 400 includes a cuff actuator 404 that can include a motor controller (e.g. the motor controller 320) and a motor assembly. In response to a signal from the cuff actuation and processing circuitry 410, the motor controller can actuate a motor included with the motor assembly. The cuff actuator 404 can cause the overlapping sleeve 402 to compress the compressible material 406 by, for example, causing at least one end of the overlapping sleeve 402 to wrap farther around a patient's limb 420. In addition, the motor assembly of the cuff actuator 404 may include one or more of the following: a cable, a pulley, and a gear assembly, such as a worm drive or any other gear assembly that can facilitate wrapping the overlapping sleeve 402 around the patient's limb 420 to compress the compressible material 406. In one embodiment, the overlapping sleeve 402 may be wrapped around itself, or around a spool using, for example, a winch mechanism.

Although not limited as such, the cuff actuator 404 of FIG. 4A is located between the two ends of the overlapping sleeve 402 such that one end of the overlapping sleeve 402 may be placed above the cuff actuator 404 and one end may be placed beneath the cuff actuator 404. To place the blood pressure cuff 400 on a patient's limb 420, the patient's limb 420 may be slipped through the blood pressure cuff 400. Alternatively, the blood pressure cuff 400 may be applied to a patient's limb by separating the two ends of the overlapping sleeve 402 and the compressible material 406. The blood pressure cuff 400 may then be wrapped around the patient's limb 420. The cuff actuator 404 may be affixed to either end of the overlapping sleeve 402 or may be affixed to another portion of the overlapping sleeve 402. Further, the cuff actuator 404 may be detachable.

In addition, the two ends of the overlapping sleeve 402 may be attached to one another using any mechanism for securing two ends of a material. For example, the overlapping sleeve 402 and the compressible material 406 may include hooks, Velcro, reusable adhesive, non-reusable adhesive, or straps, to name a few. Similarly, the two ends of the compressible material 406 may be attached to one another using any mechanism for securing two ends of a material. Further, the overlapping sleeve 402 and the compressible material 406 may use the same mechanism or different mechanisms for securing their respective ends to one another.

The blood pressure cuff 400, in whole or in part, may be disposable, reusable, or resposable. Resposable devices can include devices that are partially disposable and partially reusable. For example, the compressible material 406 may be single-use, but the overlapping sleeve 402 may be reusable. As a second example, the compressible material 406 may be resposable in that it may be reused for a single patient, but not for additional patients.

In embodiments where some or all of the blood pressure cuff 400 is reusable or resposable, portions of the blood pressure cuff 400 may be separable. Thus, in some embodiments, the overlapping sleeve 402 and the compressible material 406 are joined in a manner that enables the use of a single mechanism to secure the ends of the blood pressure cuff 400 to one another enabling the blood pressure cuff 400 to encircle the patient's limb 420. However, in some alternative embodiments, the overlapping sleeve 402 and the compressible material 406 are placed separately around the patient's limb 420. In such embodiments, the ends of the overlapping sleeve 402 and the compressible material 406 may be separately and independently secured to one another. In some embodiments, the overlapping sleeve 402 and the compressible material 406 may be secured to one another. Advantageously, in some embodiments, securing the overlapping sleeve 402 to the compressible material 406 may facilitate the application of a specific amount of pressure to the compressible material 406. Further, in some embodiments, securing the overlapping sleeve 402 to the compressible material 406 may facilitate obtaining accurate pressure readings.

The blood pressure cuff 400 may include a number of sensors to facilitate measuring a patient's blood pressure. One or more of the sensors may also be used to determine when to obtain a blood pressure reading. In the embodiment depicted in FIG. 4A, the blood pressure cuff 400 includes a pressure sensor 412 and an acoustic sensor 414. As shown in FIG. 4A, the pressure sensor 412 may be located within the compressible material 406. However, the placement of the pressure sensor 412 is not limited as such. The pressure sensor 412 may be located between the compressible material 406 and the patient's limb 420, between the compressible material 406 and the sleeve 402, or in any location that enables the pressure sensor 412 to determine a pressure reading associated with the compression of the compressible material 406.

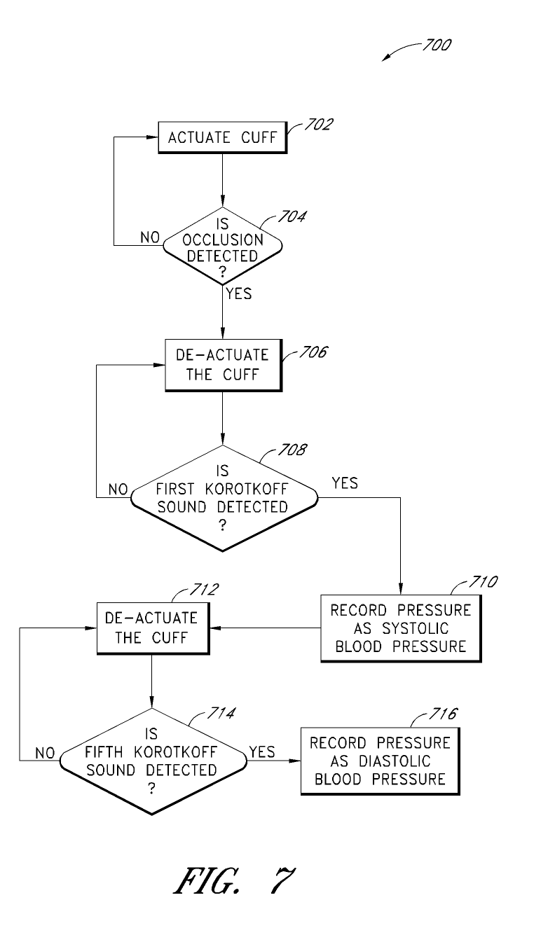

Further, as shown in FIG. 4A, the acoustic sensor 414 may be located between the compressible material 406 and the patient's limb 420 and may be configured to contact the patient's limb 420. However, the placement of the acoustic sensor 414 is not limited as such. The acoustic sensor 414 may be located within the compressible material 406, between the compressible material 406 and the sleeve 402, or in any location that enables the acoustic sensor 414 to detect biological sounds, such as Korotkoff sounds, which are described further below with reference to FIG. 6. Further, the acoustic sensor 406 may be partially or completely exposed to the patient's limb 420 enabling contact with the patient. Alternatively, the acoustic sensor 406 may not be exposed to the patient's limb 420; however, the acoustic sensor 406 may be calibrated to obtain an accurate measurement of the biological sounds without direct contact with the patient's limb 420. For example, the acoustic sensor 420 may be placed within the compressible material 406, between the compressible material 406 and the sleeve 402, or in some other location.

FIG. 4B illustrates an embodiment of a blood pressure cuff 400 with a non-overlapping sleeve 440. The non-overlapping sleeve 440 may be placed around the patient's limb 420 in a manner similar to that of the overlapping sleeve 402 as described above. However, unlike the overlapping sleeve 402, the non-overlapping sleeve 440 may compress the compressible material 406 without the ends of the sleeve overlapping one another. In the embodiment illustrated in FIG. 4B, the cuff actuator 404 may be placed beneath the ends of the non-overlapping sleeve 440. Alternatively, the cuff actuator 404 may be placed above the ends of the non-overlapping sleeve 440 or between the ends of the non-overlapping sleeve 440.

The non-overlapping sleeve 440 may be tightened using a motor assembly included with the cuff actuator 404. Similar to the overlapping sleeve 402, the non-overlapping sleeve 440 may be tightened to compress the compressible material 406 around the patient's limb 420. Further, the motor assembly may include a winch mechanism to tighten the non-overlapping sleeve 440 without the ends of the non-overlapping sleeve 440 overlapping.

### Example of an Acoustic Sensor

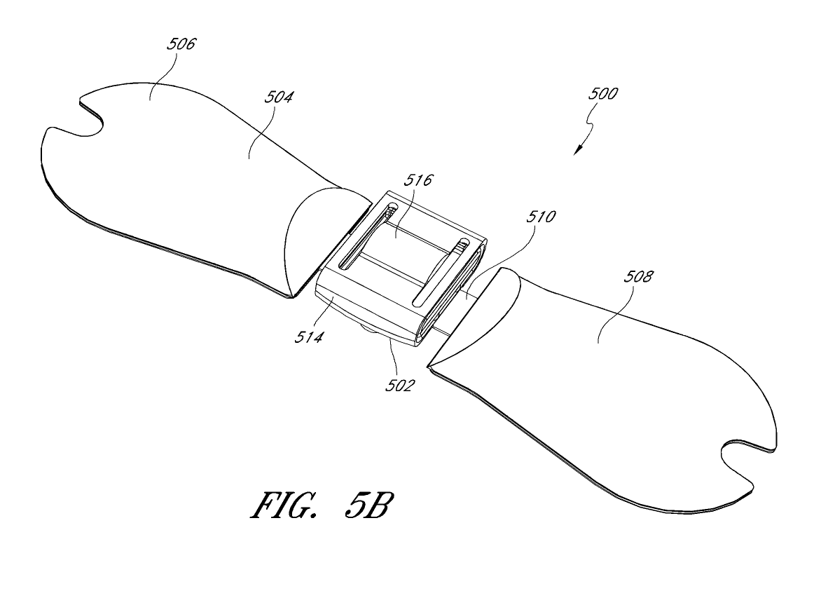

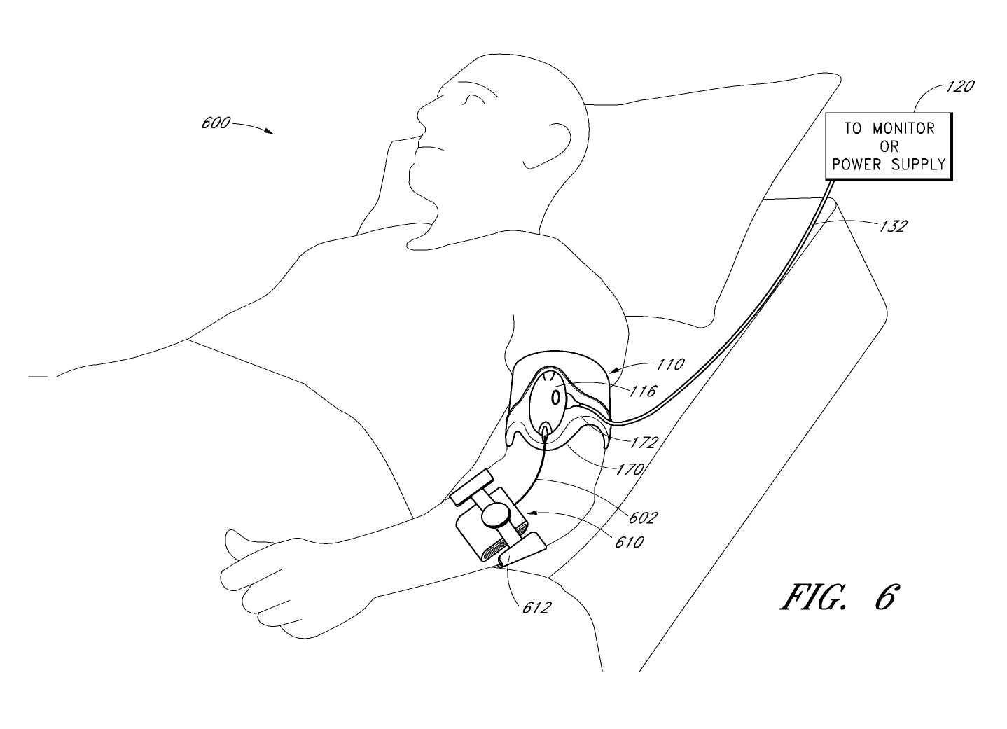

FIGS. 5A-5B illustrate top and bottom perspective views, respectively, of an acoustic sensor 500. In an embodiment, the acoustic sensor 500 includes a sensing element, such as, for example, a piezoelectric device or other acoustic sensing device. The sensing element generates a voltage that is responsive to vibrations generated by the patient, and the sensor includes circuitry to transmit the voltage generated by the sensing element to a processor for processing. In an embodiment, the acoustic sensor 500 includes circuitry for detecting and transmitting information related to biological sounds to a physiological monitor (e.g. the monitor 120 or the output device 350) and/or to the cuff actuation and processing circuitry 310 to facilitate blood pressure determination. These biological sounds may include Korotkoff sounds. Further, in some embodiments, the acoustic sensor 500 may detect heart, breathing, and/or digestive system sounds, in addition to many other physiological phenomena.

Generally, the acoustic sensor 500 is configured to be attached to a patient and the sensing element is configured to detect biological sounds (e.g. Korotkoff sounds) from a patient measurement site. The sensing element may include a piezoelectric membrane, for example, and is supported by a support structure such as a generally rectangular support frame. The piezoelectric membrane is configured to move on the frame in response to acoustic vibrations, thereby generating electrical signals indicative of the biological sounds of the patient. An electrical shielding barrier (not shown) may be included which conforms to the contours and movements of the piezoelectric element during use. Further, additional layers may be provided to help adhere the piezoelectric membrane to the electrical shielding barrier.

Embodiments of the acoustic sensor 500 may include a sensor subassembly 502. This sensor subassembly may also include an acoustic coupler, which advantageously improves the coupling between the source of the signal to be measured by the sensor (e.g., the patient's skin) and the sensing element. The acoustic coupler of one embodiment includes a bump positioned to apply pressure to the sensing element so as to bias the sensing element in tension. The acoustic coupler can also provide electrical isolation between the patient and the electrical components of the sensor, beneficially preventing potentially harmful electrical pathways or ground loops from forming and affecting the patient or the sensor.

The sensor subassembly 502 of the illustrated embodiment includes an acoustic coupler 514, which generally envelops or at least partially covers some or all of the components of the sensor subassembly 502. Referring to FIG. 5B, the bottom of the acoustic coupler 514 includes a contact portion 516 which is brought into contact with the skin of the patient.

In some embodiments, the acoustic sensor 500 may include an attachment subassembly 504. The attachment subassembly 504 may include lateral extensions symmetrically placed about the sensor subassembly 502. For example, the attachment subassembly 504 can include single, dual or multiple wing-like extensions or arms that extend from the sensor subassembly 502. In other embodiments, the sensor subassembly 502 has a circular or rounded shape, which advantageously allows uniform adhesion of the attachment subassembly 504 to an acoustic measurement site. The attachment subassembly 504 can include plastic, metal or any resilient material, including a spring or other material biased to retain its shape when bent. In the illustrated embodiment, the attachment subassembly 504 includes a first elongate portion 506, a second elongate portion 508, an elongate member 510 and a button 512. In certain embodiments the attachment subassembly 504 or portions thereof are disposable and/or removably attachable from the sensor subassembly 502. The button 512 mechanically couples the attachment subassembly 504 to the sensor subassembly 502. In some embodiments, some or all of the attachment subassembly 504 may be optional. For example, in embodiments of the compressible material 406 that may include the acoustic sensor, the attachment subassembly 504 may be unnecessary.

As is described in further detail with respect to FIG. 6 and FIG. 7, the acoustic sensor 500 may be used to facilitate determining when to measure various blood pressure readings. For example, the acoustic sensor can be used to detect the first Korotkoff sound indicating when to measure the systolic blood pressure of a patient. Additional examples of acoustic sensors that can be used herein are described in U.S. application Ser. No. 12/643,939, filed Dec. 21, 2009, titled “Acoustic Sensor Assembly,” which is hereby incorporated by reference in its entirety.

### Third Example of a Patient Monitoring System

FIG. 6 illustrates another embodiment of a patient monitoring system 600. The features of the patient monitoring system 600 can be combined with any of the features of the systems described above. Likewise, any of the features described above can be incorporated into the patient monitoring system 600. Further, elements of FIG. 6 that share reference numerals with elements of FIG. 1 and/or FIG. 2 may be configured similarly. FIG. 6 shows the cuff 110 of FIG. 1 in combination with a separate acoustic sensor 610.

The patient monitoring system 600 can include an acoustic sensor 610. This acoustic sensor 610 can include any type of acoustic sensor configured to detect biological sounds as described above. Further, in the depicted embodiment, the acoustic sensor 610 can be configured to provide a signal associated with the detection or lack of detection of biological sounds to the cuff 110 or the patient device 116. Further, the signal may be provided to the cuff measurement and control system 300 and/or the cuff actuation and processing circuitry 310. In some embodiments, the acoustic sensor 610 may provide the signal to the monitor 120. Communication with the acoustic sensor 610 may occur wirelessly or via wire 602.

The acoustic sensor 610 may be attached to the patient using any mechanism appropriate for the selected placement of the acoustic sensor 610. For example, in the depicted embodiment of FIG. 6, the acoustic sensor 610 may be attached using butterfly straps or elongated straps that can wrap around a patient's limb. As additional examples, the acoustic sensor 610 may be attached to the patient using an adhesive or may be attached to the blood pressure cuff 110 using straps, a clip, or the like. Further, although depicted apart from the blood pressure cuff 110, the acoustic sensor 610 may be located adjacent to the blood pressure cuff 110. Alternatively, the acoustic sensor 610 may be located, at least in part, beneath the blood pressure cuff 110 between the cuff and the portion of the patient wrapped by the cuff. Additionally, the placement of the acoustic sensor 610 is not limited to the distal portion of the patient limb. The acoustic sensor 610 may be placed virtually anywhere on the patient including, for example: between the blood pressure cuff 110 and the distal portion of the patient's limb; between the blood pressure cuff 110 and the proximal portion of the patient's limb; beneath the blood pressure cuff 110, at least in part; or any other portion of the patient from which the acoustic sensor 610 can detect biological sounds.

In some embodiments, the patient monitoring system 600 determines when to obtain a systolic blood pressure reading and/or when to obtain a diastolic blood pressure reading based, at least in part, on information related to a patient's biological sounds provided by the acoustic sensor 610. As previously mentioned, these biological sounds may include Korotkoff sounds. Korotkoff sounds can include sounds that occur during de-occlusion of a blood vessel. To hear the Korotkoff sounds, a blood vessel is occluded so that blood can no longer flow through the vessel past the point of occlusion and then the blood vessel is de-occluded allowing blood to flow again. As the blood passes through the blood vessel, biological sounds may be detected. When the amount of pressure applied to the blood vessel is reduced to a level equal to a patient's systolic blood pressure, the first Korotkoff sound is produced. As pressure is further reduced, additional Korotkoff sounds can be detected. The diastolic pressure may be taken when the fourth Korotkoff sound is barely audible, or when the fifth Korotkoff sound is detected, which may be silence or no sound.

Further, in some embodiments, the acoustic sensor 610 can facilitate determining a pulse-wave transit time (PWTT), which can be used to trigger the blood pressure cuff 110 as further described below with reference to FIG. 9. Additional examples, of blood pressure measurement systems, including systems with acoustic sensors and systems capable of measuring PWTT, that can be used herein are described in the following provisional applications, each of which is hereby incorporated by reference in its entirety: U.S. Provisional Application No. 61/469,511, filed Mar. 30, 2011, titled “Non-Invasive Blood Pressure Measurement System” and U.S. Provisional Application No. 61/366,862, filed Jul. 22, 2010, titled “System for Triggering Non-Invasive Blood Pressure Device.”

### Example of a Blood Pressure Measurement Process

FIG. 7 illustrates a flow diagram for one embodiment of a blood pressure measurement process 700. The process 700 may be performed by any cuff capable of determining a patient's blood pressure. Advantageously, in some embodiments, the process 700 can be performed, at least in part, by a non-inflatable blood pressure cuff, such as the blood pressure cuff 110 or 400. Although any number of systems, in whole or in part, can implement the process 700, to simplify discussion, portions of the process 700 will be described with reference to particular systems.

At block 702, the blood pressure cuff 110, for example, is actuated. The actuation may be in response to a command from the monitor 120, the cuff measurement and control system 300, or the cuff actuation and processing circuitry 310. Further, the blood pressure cuff 110 may be an oscillatory cuff that is actuated at pre-determined time intervals. The blood pressure cuff 110 may also be an automatic blood pressure cuff that is actuated in response to a physiological measurement (e.g. a PWTT or an ECG measurement). In some embodiments, a user (e.g. a healthcare worker) may actuate the blood pressure cuff 110. Actuating the blood pressure cuff 110 can include the motor controller 320 actuating a motor assembly to cause the sleeve 172 to compress the compressible material 170. The rate and level of compression of the compressible material 170 may be determined by one or more of the cuff actuation and processing circuitry 310 and the motor controller 320. Further, the rate and level of compression may be based, at least in part, on measurements obtained by one or more of the pressure sensor 330, the acoustic sensor 340, the acoustic sensor 610, the optical sensor 202, and any other sensors that may be associated with the blood pressure cuff 110 and that can determine physiological parameters associated with the patient.

At decision block 704, the blood pressure cuff 110 determines whether occlusion of a blood vessel being measured (e.g. the brachial artery) is detected. Determining whether the blood vessel is occluded may include determining if the level of occlusion in the blood vessel satisfies a threshold. This threshold may vary based on the blood vessel being occluded. Further, the threshold may vary based on the individual patient including the patient's overall condition and/or the patient's condition at the time of blood pressure measurement. The level of occlusion may be determined based on the readings of one or more physiological sensors. For example, the level of occlusion may be determined based on biological sounds measured by the acoustic sensor 340 or 610. If no sound is detected by the acoustic sensors, then it may be determined that the blood vessel is fully occluded. Further, the detection of turbulence followed by silence may be used to detect the level of occlusion in the blood vessel and/or when to identify a blood pressure value. As a second example, the level of occlusion may be determined by the optical sensor 202. The optical sensor 202 may be used to obtain photoplethysmograph (PPG) measurements. Attenuation, peaks, drop in amplitude, or disappearance of PPG readings may be used separately or in combination to determine the level of in the measured blood vessel. In another example, the level of occlusion may be determined by measuring PWTT between, for example, the heart and a portion of the patient's limb beyond the occluded vessel. If the PWTT becomes undetectable, then the blood vessel may be occluded. Further, changes to the PWTT may indicate a change in the level of occlusion of the blood vessel.

If occlusion in the blood vessel is not detected, or if the level of occlusion does not satisfy a threshold, the blood pressure cuff 110 may continue to actuate the cuff at block 702. In some embodiments, the blood pressure cuff 110 may alter the rate and level of compression of the compressible material 170 based on the level of occlusion detected. For example, if the blood vessel is determined to be 90% occluded, the blood pressure cuff 110 may reduce the rate of compression, or, alternatively, increase the level of compressive force being applied by the sleeve 172 to the compressible material 170.

If the blood pressure cuff 110 determines that the blood vessel is occluded, or that the level of occlusion satisfies a threshold, the blood pressure cuff 110 is de-actuated at block 706. The blood pressure cuff 110 may be passively de-actuated by, for example, ceasing to apply pressure to the compressible material 170 thereby causing the occlusive pressure applied to the blood vessel to decrease. Alternatively, the blood pressure cuff 110 may be actively de-actuated by loosening the sleeve 172 and/or the compressible material 170 by, for example, causing the motor controller 320 to operate the motor assembly in reverse. In some embodiments, the cuff measurement and control system 300 may control the rate at which the blood pressure cuff 110 is de-actuated by controlling the rate at which compression of the compressible material 170 is reduced.

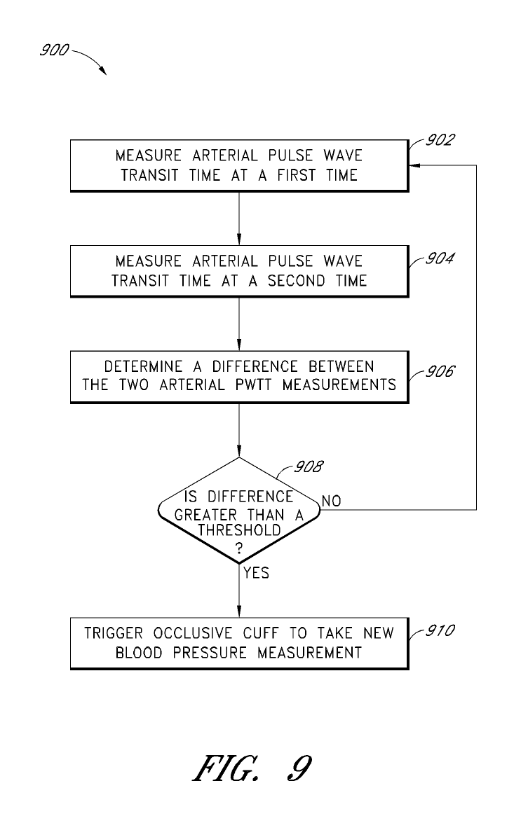

Using, for example, the acoustic sensor 340 or 610, the blood pressure cuff 110 determines whether the first Korotkoff sound is detected at decision block 708. If not, the blood pressure cuff 110 is further de-actuated at block 706 until the first Korotkoff sound is detected at decision block 708. At block 710, the blood pressure cuff 110 using, for example, the pressure sensor 330, can determine the patient's systolic pressure for the blood vessel being measured. The systolic pressure can be determined based on the pressure applied to the compressible material 170 as detected by the pressure sensor 330 and a calibration curve. This calibration curve is described in more detail below with respect to FIG. 8.

At block 712, the blood pressure cuff 110 is further de-actuated. Further de-actuation of the blood pressure cuff 110 may occur by further active decompressing of the compressible material 170. Alternatively, the de-actuation may occur as a result of the passage of time due to the cessation of the application of pressure on the compressible material 170.

At decision block 714, using, for example, the acoustic sensor 340 or 610, the blood pressure cuff 110 determines whether the fifth Korotkoff sound is detected. In some embodiments, the blood pressure cuff 110 determines at decision block 714 whether the fourth Korotkoff sound is detected. Alternatively, the blood pressure cuff 110, using the acoustic sensor 340, may be configured to detect the fourth or fifth Korotkoff sounds based on the patient whose blood pressure is being measured. For example, if the patient is a child, the blood pressure cuff 110 may be configured to determine whether the fourth Korotkoff sound is detected at decision block 714, and if the patient is an adult, the blood pressure cuff 110 may be configured to determine whether the fifth Korotkoff sound is detected at decision block 714. In some instances, the detected sounds may be periodic, in other instances the detected sounds may be aperiodic. In other instances, silence may be detected and treated as a “sound” for the purpose of obtaining a blood pressure reading.

If the fifth Korotkoff sound is not detected at decision block 714, the blood pressure cuff is further de-actuated at block 712 until the fifth Korotkoff sound is detected at decision block 714. At block 716, the blood pressure cuff 110 using, for example, the pressure sensor 330, can determine the patient's diastolic pressure for the blood vessel being measured. The diastolic pressure can be determined based on the pressure applied to the compressible material 170 as detected by the pressure sensor 330 and the relation between the applied pressure and a blood pressure value on the calibration curve.

In certain embodiments, the various Korotkoff sounds are detected by, for example, the acoustic sensor 340 based on the turbulence, or lack thereof, that occurs as blood flows through a blood vessel that is de-occluded after having been occluded to at least a threshold degree. This threshold is often associated with the level of occlusion where no blood flows past the point or region of occlusion, but, in some cases, the threshold may allow for some blood flow. As the blood vessel is de-occluded, the blood may begin to flow through the blood vessel past the point of occlusion. This blood flow may be in spurts and occurs as the pressure in the blood vessel rises above the pressure of the cuff 110 and then falls as the blood passes the point of occlusion. Further, the blood flow may result in turbulence which produces an audible sound. As the pressure created by the cuff 110 continues falling, thumping sounds may continue to be heard. Eventually, as the pressure created by the cuff 110 subsides, the sounds created by the turbulent blood flow decrease and eventually disappear as the cuff 110 ceases to restrict the blood flow in the blood vessel. Once the blood flow is smooth, the turbulent sounds are no longer detected.

Generally, the first Korotkoff sound detected as the blood begins flowing through the previously occluded blood vessel is associated with the systolic pressure. This first Korotkoff sound may sound like clear, tapping repetitive sounds if heard via a stethoscope. The second Korotkoff sound can be described as murmurs heard between the systolic and diastolic pressures. The third Korotkoff sound may be a loud, crisp tapping sound. The fourth Korotkoff sound is generally associated with the diastolic blood pressure and may sound like thumping and muting. The fifth Korotkoff sound is silence, which occurs when the pressure of the cuff 110 falls below the diastolic blood pressure. In some instances, the fifth Korotkoff sound is associated with the diastolic blood pressure.

In some embodiments, at least one of the cuff 110, the acoustic sensor 340, the monitor 120, and the cuff actuation and processing circuitry 310 can identify the Korotkoff sounds by processing the signals captured by the acoustic sensor 340 and comparing the processed signals to one or more spectral signatures. These spectral signatures are representations of the sounds described above and can be interpreted and processed by a signal processor or digital signal processor. Further, the spectral signatures can be associated with particular sounds and/or particular Korotkoff sounds. For example, the tapping repetitive sounds may be represented by a periodic waveform. As a second example, the thumping sound may be detected as a dense waveform concentrated around a particular set of frequencies. In some embodiments, the spectral signatures are identified based on the signals as detected by the acoustic sensor 340. Alternatively, the spectral signatures are associated with processed versions of the signals detected by the acoustic sensor 340. For example, the detected signals may be filtered and the filtered detected signals may be compared to the spectral signatures to determine the detected Korotkoff sounds, or other sounds indicative of when to obtain a blood pressure measurement.

The systolic blood pressure and diastolic blood pressure, as detected at block 710 and 716 respectively, may be presented to a user (e.g. a patient or a healthcare worker) via, for example, the output device 350 or the patient monitor 120. Further, the systolic blood pressure and diastolic blood pressure may be recorded by the monitor 120, the patient device 116, or any other medical recordation or monitoring system. Moreover, the blood pressure readings may be recorded to any repository that can store information associated with the patient.

In some embodiments, the blood pressure cuff 110 or the monitor 120, for example, may alert the user of the status of a patient in response to the systolic and/or diastolic blood pressure satisfying a threshold. This alert may be visual or auditory. Further, the alert may be presented via email, text, or the activation of an alarm. In some embodiments, the status of the patient may be presented via the wellness monitor 290. For example, if there is a sudden drop in blood pressure, the wellness monitor 290 may activate a light emitting diode (LED) on the blood pressure cuff 110 or the monitor 120, or may adjust the color of the LED. Further, in some embodiments, additional medical systems may be activated in response to the blood pressure readings. For example, medication may be automatically administered, or additional monitoring systems may be activated.

### Example of a Calibration Curve

FIG. 8 illustrates an embodiment of a calibration curve 800. One or more systems or subsystems associated with the patient monitoring system may use the calibration curve 800 to facilitate determining blood pressure in a blood vessel of a patient. For example, the cuff actuation and processing circuitry 310 of the blood pressure cuff 110 or the monitor 120 may use the calibration curve 800 to facilitate identification of a patient's blood pressure. Although any number of systems may use the calibration curve 800 to facilitate identifying the patient's blood pressure, to simplify discussion, the calibration curve 800 will be described as being used by the cuff actuation and processing circuitry 310. Further, the depiction of the calibration curve 800 as a graph is for illustrative purposes. The calibration curve 800 may be represented in any format that facilitates the measurement of the patient's blood pressure. For example, the information presented by the calibration curve 800 may be in tabular form or in a machine-readable form stored in memory. Further, although the pressure axes as depicted in FIG. 8 are in units of millimeters of mercury (mmHg), the pressure axes and the calibration curve 800 are not limited as such. For example, the pressure values may be in terms of Torr, atm or psi, to name a few.

In one embodiment, the calibration curve 800 may be used to identify the blood pressure in the blood vessel of the patient at one or more pre-defined points in time corresponding to the level of occlusion of the patient's blood vessel. Although not limited as such, these pre-defined points in time may be associated with the occurrence of the Korotkoff sounds that may occur as the patient's blood vessel is de-occluded. For example, to identify the systolic blood pressure value, the cuff actuation and processing circuitry 310 using, for example, the pressure sensor 330 can identify a pressure value associated with the amount of compression applied by the sleeve 172 to the compressive material 170 during the occurrence of the first Korotkoff sound. As an example, this pressure value may be represented by the sensor pressure value 802 on the X-axis in FIG. 8, which corresponds to the point 804 on the calibration curve. Using the point 804, the cuff actuation and processing circuitry 310 can identify the systolic blood pressure represented by the pressure value 806 on the y-axis in FIG. 8.

The x-axis of the calibration curve 800 can represent the pressure value of the blood pressure cuff 110 (e.g. the pressure associated with the amount of compression applied by the sleeve 172 to the compressive material 170). Further, the value on the x-axis may be associated with a level of occlusion of a blood vessel. The y-axis of the calibration curve 800 can represent blood pressure values for given populations. For example, the values on the y-axis may be based on the blood pressure of a sample population. In some embodiments, the values on the y-axis may be based on values obtained from another portion of the patient. For example, when the blood pressure cuff 110 is attached to the left arm, the y-axis may be based on blood pressure values measured in the right arm, either historically, or at substantially the same time as measurements in the left arm. Further, in some embodiments, the y-axis may be based on historical values obtained from the same portion of the patient as is currently being measured.

### Example Process for Triggering an Occlusive Blood Pressure Measurement

FIG. 9 illustrates an embodiment of a process 900 for triggering an occlusive blood pressure measurement. This process 900 can be implemented by any system capable of making PWTT measurements and blood pressure measurements. For example, the process 900 can be implemented by system 200 described above. Advantageously, in certain embodiments, the process 900 can determine, based at least partly on non-invasive PWTT measurements, whether to trigger an automatic occlusive cuff. As a result, continuous or substantially continuous monitoring of a user's blood pressure can occur, allowing the frequency of occlusive cuff measurements to potentially be reduced.

At block 902, a first arterial PWTT measurement is determined at a first point in time. The arterial PWTT can be determined using any number of techniques, such as by calculating a patient's Pre-Ejection Period (PEP) and by compensating an overall PWTT value with the PEP. Additional examples of PWTT measurement techniques that can be used herein are described in U.S. Provisional Application No. 61/366,862, referred to above. Similarly, a second arterial PWTT measurement is taken at a second point in time at block 904. These PWTT measurements can be taken from successive heart beats in one embodiment. In another embodiment, the first and second arterial PWTT values each represent PWTT values averaged over multiple heartbeats.

At block 906, a difference between the two arterial PWTT measurements is determined. It is then determined at decision block 908 whether the difference between the two measurements is greater than a threshold. A difference greater than a threshold can be indicative of a change in a patient's blood pressure. Therefore, if the difference is greater than the threshold, an occlusive cuff is triggered to take a new blood pressure measurement at block 910. If the difference is not greater than the threshold, then the process 900 loops back to block 902. Effectively, the process 900 therefore can trigger occlusive cuff measurements when the threshold is exceeded and can continue monitoring PWTT measurements otherwise. In some embodiments, the occlusive cuff may be triggered to take the new blood pressure measurement if the difference satisfies a threshold.

In certain embodiments, the process 900 analyzes changes in PWTT measurements using an absolute difference technique or a moving difference technique. With the absolute difference technique, the process 900 measures the PWTT at a first fixed time. Subsequent PWTT measurements (e.g., the second measurement at block 904) are compared to the initial PWTT at the first fixed time to determine whether the difference between these measurements exceeds a threshold. With the moving difference technique, the first and second PWTT measurements are compared for successive points in time. The first PWTT measurement is therefore not taken at a fixed time but instead changes over time. Thus, the moving difference technique can approximate a derivative of the PWTT measurements. The moving difference can be compared to a threshold at block 908. An advantage of using the moving difference technique is that it can potentially ignore drifts in PWTT measurements due to calibration changes or other errors.

Thus, in certain embodiments, the process 900 can refrain from triggering an occlusive cuff until the non-invasive measurement differs enough to trigger such a measurement. Advantageously, in certain embodiments, the process 900 can therefore allow a user to postpone the discomfort and potential physiological damage associated with occlusive blood pressure measurements, while the non-invasive measurement (PWTT) is within a certain tolerance.

Although the PWTT measurements have been described herein as being used to trigger an occlusive cuff, in certain embodiments the PWTT measurements can additionally, or alternatively, be used to derive an estimate of blood pressure. A calibration function or curve can be determined that maps PWTT measurements to blood pressure values. The slope and intercept of the calibration curve can be determined experimentally.

### Second Example of a Cuff Measurement and Control System

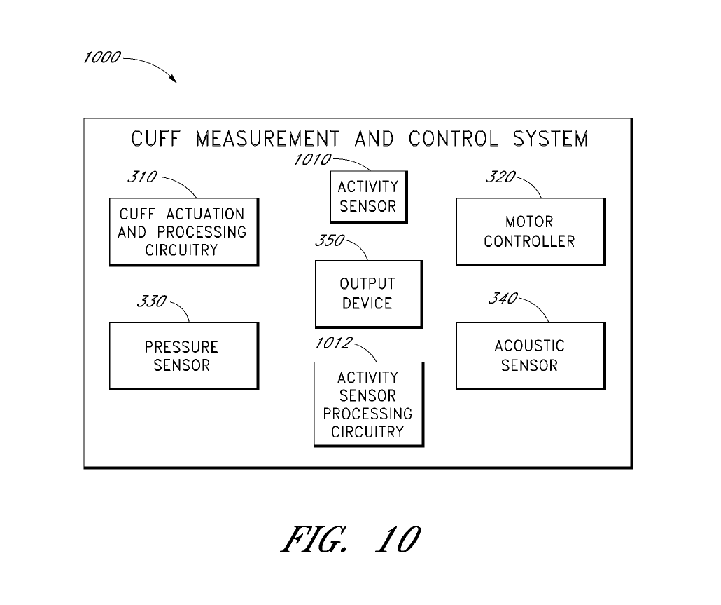

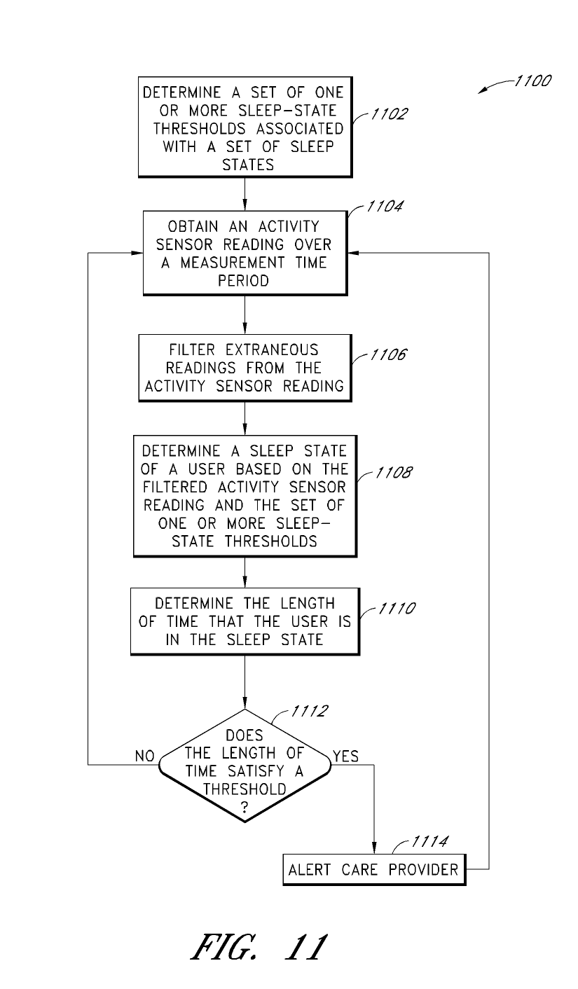

FIG. 10 illustrates another embodiment of a cuff measurement and control system 1000. The cuff measurement and control system 1000 can include any system for controlling a blood pressure cuff (e.g. cuff 110) and for obtaining, using the blood pressure cuff, physiological information to be provided to a healthcare worker and/or to another system, such as the monitor 120. Further, in certain embodiments, the cuff measurement and control system 1000 can include some or all of the systems and/or embodiments described above with relation to the cuff measurement and control system 300. Similarly, in certain embodiments, like-numbered elements of the cuff measurement and control system 1000 may include some or all of the embodiments described above with reference to like-numbered elements of the cuff measurement and control system 300.

In addition to the aforementioned like-numbered elements (e.g., pressure sensor 330, acoustic sensor 340, etc.), the cuff measurement and control system 1000 includes an activity sensor 1010 and activity sensor processing circuitry 1012. The activity sensor 1010 can generally include any type of sensor for measuring activity, movement, or motion. Further, the activity sensor 1010 readings can be used to determine types of movement and the intensity of movement. In some cases, using the activity sensor 1010, it is possible to determine a sleep state of a user. In certain cases, the activity sensor processing circuitry 1012 can use the readings of the activity sensor 1010 to determine the probability that a user is in a particular sleep state. The activity measurement may be an instantaneous measurement or a measurement obtained over a period of time (e.g., 30 seconds, 5 minutes, a hour, etc.). The activity sensor 1010 can include a piezoelectric sensor, a piezoelectric accelerometer, an actigraph (or actograph), an electroencephalogram (EEG), an electromyogram (EMG), an electro-oculogram (EOG), or any other type of sensor that is capable of measuring the activity or motion of a user. For example, in one embodiment, the activity sensor 1010 is a piezoelectric sensor or a resistive stress sensor integrated with the blood pressure cuff described above. In addition, in some embodiments, any of the pressure sensors or acoustic sensors described above can be used as the activity sensor 1010.

The cuff measurement and control system 1000 may be configured to obtain continuous activity measurements using the activity sensor 1010 of a user (e.g., a patient). For instance, the cuff measurement and control system 1000 can determine from a piezoelectric or other activity sensor 1010 whether the patient has moved recently, and if not, may indicate that the patient is sleeping. Alternatively, the cuff measurement and control system 1000 may be configured to obtain sleep state measurements at particular points in time or over particular periods of time by, for example, measuring activity over the particular points in time or over the particular periods of time. In other cases, the cuff measurement and control system 1000 may determine sleep states in response to a user (e.g., a care provider) initiating a sleep state determination process (e.g., the sleep related illness detection process 1100 of FIG. 11).

The activity sensor processing circuitry 1012 can include any type of processor or circuitry that can process the measurements obtained by the activity sensor 1010. The processing of the measurements can include filtering the measurements to remove noise or readings that do not satisfy particular thresholds or conditions associated with detecting sleep states. In addition, in some cases, the activity sensor processing circuitry 1012 can determine the type of activity or movement of a user based on the readings of the activity sensor 1010. Moreover, in some cases, the activity sensor processing circuitry 1012 can determine the intensity of activity or movement of the user. Further, the activity sensor processing circuitry 1012 can compare one or more activity sensor 1010 measurements over a period of time against one or more thresholds to facilitate determining a user's sleep state. Based, at least in part, on the comparisons, the activity sensor processing circuitry 1012 can determine a probability that the user may have a sleep condition, atypical sleep pattern compared to guidelines established by one or more medical professionals, sleep-related illness, or sleep-related symptom of some identified or unidentified illness.

Although FIG. 10 illustrates the activity sensor 1010 and the activity sensor processing circuitry 1012 as separate components, in some embodiments, the activity sensor 1010 and the activity sensor processing circuitry 1012 may be combined into a single component. Alternatively, the activity sensor processing circuitry 1012 may be divided into multiple components. For example, the cuff measurement and control system 1000 may include a component to filter the activity sensor 1010 readings, a separate component to perform threshold comparisons of activity sensor 1010 readings to identify a sleep state, and a separate component to calculate the probability that the user has a sleep condition.

In some embodiments, the monitor 120 may include the activity sensor processing circuitry 1012. In such cases, the activity sensor 1010 may obtain activity readings and then provide the readings to the monitor 120 for further processing.

### Example of a Sleep-Related Illness Detection Process

FIG. 11 illustrates a flow diagram for one embodiment of a sleep detection process 1100. The process 1100 may be performed by any cuff that includes or that communicates with an activity sensor 1010. Advantageously, in some embodiments, the process 1100 can be performed, at least in part, by a cuff that includes the cuff measurement and control system 1000, an activity sensor 1010, and activity sensor processing circuitry 1012. In some cases, the process 1100 can be performed, at least in part, by any cuff that communicates with a monitor that includes activity sensor processing circuitry 1012. Although any number of systems, in whole or in part, can implement the process 1100, to simplify discussion, portions of the process 1100 will be described with reference to particular systems. The process 1100 can be used to detect when a patient is sleeping for a number of purposes including, for example, the purpose of a sleep study, for detecting sleep-related illness, or for other patient monitoring purposes. Further, the process 1100 may be used to determine when a user is awake so as, for example, to provide the user with medicine or to determine when a care provider can interact with the user without waking the user.

The process 1100 begins at block 1102 where, for example, the activity sensor processing circuitry 1012 determines a set of one or more sleep-state thresholds associated with a set of sleep states. The sleep-state thresholds may be based, at least in part, on a set of sleep state definitions specified by one or more medical practitioners or medical associations (e.g., the American Academy of Sleep Medicine). In some cases, the sleep-state thresholds may be based, at least in part, on a specific patient or set of users who share at least one characteristic with the patient (e.g., users of a particular age, users with a particular identified disease or condition, etc.). Each sleep state may correspond to one or more sleep-state thresholds. Thus, in some cases, a sleep state may be defined based on whether it satisfies a sleep-state threshold or is between two sleep-state thresholds.

Sleep may be divided into different states or stages. The number of stages may vary based on the definition of each stage. One common categorization of sleep stages includes Rapid Eye Movement (REM) and Non-Rapid Eye Movement (NREM) stages. NREM sleep may further be divided into three stages including N1, N2, and N3. In some cases, the time period when the body prepares for sleep may be considered an additional sleep stage and is sometimes termed “waking.”

In some cases, it is possible to identify a sleep stage of a user based on body movements. Each sleep stage may be associated with a different frequency and/or intensity (e.g., rapidity of movement, level of change in body position, etc.) of body movements. Thus, a patient who moves at least a threshold amount and/or with a threshold intensity may be determined to be in a particular sleep stage. The body movement can be measured over a measurement time period, such as about 30 seconds, less than or more than 30 seconds (e.g., a few minutes), or the like. For example, the activity sensor processing circuitry 1012 can detect arousals from sleep by determining that movement detected by the activity sensor 1010 occurred over a short period of time, such as about 3 seconds to about 15 seconds. The duration of the arousal period can also reflect the stage of sleep that the patient is in. In some cases, a sleep stage is identified based on an amount of movement that satisfies a threshold over the measurement time period. In other cases, a sleep stage may be identified based on movement that satisfies a threshold for an instantaneous moment during the measurement time period.

The activity sensor processing circuitry 1012 can evaluate the intensity and duration of the measurements obtained by the activity sensor 1010 to determine whether any of the above thresholds have been satisfied. The activity sensor processing circuitry 1012 can also evaluate the measurements from the activity sensor 1010 based on patterns of movement to determine whether a patient is sleeping. As many people move at least somewhat in their sleep, typical movement patterns of sleep can be evaluated against the activity sensor 1010 data to determine whether the movement corresponds to expected sleep movement. One or more patterns can be stored related to relaxed awake movements that may be mistaken for sleep (such as television watching). A patient may have relatively low movement during such activities, but this movement may differ than the pattern of movement occurring during sleep. The activity sensor processing circuitry 1012 can determine whether the patient's movement corresponds to relaxed awake activities, such as watching television, or actual sleep.

At block 1104, the activity sensor 1010 obtains an activity reading or measurement over a measurement time period. Extraneous readings may be filtered from the activity sensor 1010 reading at block 1106. The extraneous readings can include noise, vibrations, signals associated with non-sleep related movements (e.g., measurement of blood pressure via a cuff), or activity sensor 1010 measurements that exist for less than a threshold time period. In some embodiments, the block 1106 may be optional.

At block 1108, the activity sensor processing circuitry 1012 determines a sleep state of a user based on the filtered (or unfiltered in some cases) activity sensor reading and the set of one or more sleep stage thresholds identified at the block 1102. In some cases, the sleep state is determined based on a set of activity sensor readings. Determining the sleep state can include identifying the sleep stage thresholds satisfied by the activity sensor readings and the contiguous length of time or, in some cases, the total amount of time regardless of contiguity that the activity sensor readings satisfy the identified sleep stage thresholds. Further, in some cases, determining the sleep state includes determining the probability that the user is in the particular sleep state.

At block 1110, the activity sensor processing circuitry 1012 determines the length of time that the user is in the sleep state identified at the block 1108. The activity sensor processing circuitry 1012 determines at decision block 1112 whether the length of time that the user is in the sleep state satisfies a threshold. This time threshold may be a maximum threshold or a minimum threshold and may depend on the particular sleep state being evaluated. Further, in some cases the time threshold may be related to the length of time that the user is in a different sleep state.

If the length of time that the user is in the sleep state identified at the block 1108 does not satisfy the threshold, the activity sensor 1010 may continue obtaining activity sensor readings at the block 1104. Alternatively, the process 1100 may end. If the length of time that the user is in the sleep state identified at the block 1108 does satisfy the threshold, the cuff measurement and control system 1000 using, for example, the output device 330 alerts a care provider, or other user, that the user whose sleep is being analyzed has a probability of a sleep-related illness, anomaly, or sleep-related symptoms related to an illness. This probability may be based on the determinations at decision block 1112 and 1110. In some cases, the activity sensor processing circuitry 1012 may determine a specific probability that the user has a sleep-related illness based on the determinations at the blocks 1110 and 1112. In other cases, the activity sensor processing circuitry 1012 may determine that the user is more likely to have a sleep-related illness than not have a sleep-related illness.

In some embodiments, the block 1114 may include recording the measurements of the activity sensor 1010 in a memory or database associated with the cuff or the cuff measurement and control system 1000. Further, alerting the care provider may include providing the activity sensor readings to the care provider either automatically or upon request. In some embodiments, the alerts may be provided using the monitor 120. The block 1114 can also include presenting the user with a history of their sleep state upon request, or upon identifying that the user has awakened from a sleep. In some embodiments, the block 1114 is optional.

In some embodiments, the decision block 1112 may include additional or alternative determinations to decide whether the user may have a sleep-related illness. For example, the decision block 1112 may include determining whether the frequency or the length of time that the user is in the sleep state compared to how long or how often the user is in another sleep state satisfies a threshold or ratio. In some cases, a user who fails to enter a particular sleep state or spends proportionately less time in one sleep state compared to another sleep state may be associated with a particular probability of a sleep problem or illness. Thus, in certain embodiments, it is advantageous to measure the length of time that a user is in one sleep state and the comparative length of time that the user is in different sleep states.

### Terminology

The modules described herein of certain embodiments can be implemented as software modules, hardware modules, or a combination thereof. In general, the word “module,” as used herein, can refer to logic embodied in hardware or firmware or to a collection of software instructions executable on a processor. Additionally, the modules or components thereof can be implemented in analog circuitry in some embodiments.

Conditional language used herein, such as, among others, “can,” “could,” “might,” “can,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out all together (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A blood pressure device comprising:
a blood pressure cuff configured to selectively occlude and de-occlude a blood vessel of a patient without inflation of the blood pressure cuff;
an acoustic sensor configured to detect a biological sound of the patient responsive to de-occlusion of the blood vessel, the biological sound reflecting a measurement time at which a blood pressure measurement should be taken; and
a pressure sensor configured to output a pressure signal responsive to actuation of the blood pressure cuff, wherein the pressure signal at the measurement time is indicative of a blood pressure of the patient.

2. The blood pressure device of claim 1, further comprising an optical sensor configured to detect reduced pulsatile blood flow in an extremity of the patient, wherein said reduction in pulsatile blood flow reflects substantial occlusion of the blood vessel.

3. The blood pressure device of claim 2, wherein the blood pressure cuff is configured to de-occlude the blood vessel responsive to detection of the reduced pulsatile blood flow.

4. The blood pressure device of claim 1, wherein the biological sounds comprise one or more Korotkoff sounds.

5. The blood pressure device of claim 4, wherein the measurement time corresponds to a first Korotkoff sound.

6. The blood pressure device of claim 4, wherein the measurement time corresponds to a fifth Korotkoff sound.

7. The blood pressure device of claim 1, further comprising a processor configured to calculate the blood pressure of the patient responsive to the pressure signal.

8. The blood pressure device of claim 1, further comprising an activity sensor configured to determine a sleep state measurement of the patient.

9. The blood pressure device of claim 8, wherein the activity sensor comprises a piezoelectric sensor.

10. The blood pressure device of claim 1, further comprising a processor configured to determine a sleep state of the patient based on a set of sleep state measurements.

11. The blood pressure device of claim 1, wherein the pressure sensor is an activity sensor.

12. A blood pressure device comprising:
a compressible material configured to be placed around a limb of a patient;
a sleeve disposed at least partially around the compressible material and configured to compress the compressible material; and
a pressure sensor configured to output a pressure signal responsive to compression of the compressible material, wherein the pressure signal is configured to reflect a blood pressure of the patient.

13. The blood pressure device of claim 12, wherein the sleeve comprises a motor assembly configured to cause the sleeve to compress the compressible material.

14. The blood pressure device of claim 12, wherein the compressible material comprises a gel material.

15. The blood pressure device of claim 12, wherein the sleeve comprises an at least partially-rigid material.

16. The blood pressure device of claim 12, further comprising an acoustic sensor configured to detect biological sounds of the patient responsive to compression of the compressible material.

17. A method for using a blood pressure device, the method comprising:
causing a non-inflatable blood pressure cuff to occlude a blood vessel of a patient;
causing the non-inflatable blood pressure cuff to de-occlude the blood vessel subsequent to said occlusion;
detecting, with an acoustic sensor, a biological sound responsive to said de-occlusion of the blood vessel; and
taking a blood pressure reading responsive to detection of the biological sound.

18. The method of claim 17, further comprising confirming that the blood vessel is substantially occluded prior to causing the non-inflatable blood pressure cuff to de-occlude the blood vessel.

19. The method of claim 17, wherein the biological sound comprises a Korotkoff sound.

20. The method of claim 17, further comprising positioning the acoustic sensor at least partially under the blood pressure cuff.
